(19) United States
(12) Patent Application Publication
(10) Pub. No.: US 2022/0296865 A1
(43) Pub. Date: Sep. 22, 2022

(54) PERCUTANEOUS POTTS SHUNT DEVICES AND RELATED METHODS

(71) Applicant: Transmural Systems LLC, Andover, MA (US)

(72) Inventors: Nasser Rafiee, Andover, MA (US); Stuart MacDonald, Andover, MA (US); Koosha Rafiee, Andover, MA (US); Mai Le Diep, Andover, MA (US)

(21) Appl. No.: 17/717,389

(22) Filed: Apr. 11, 2022

Related U.S. Application Data
(63) Continuation-in-part of application No. 16/264,402, filed on Jan. 31, 2019.
Application No. 16/264,402 is a continuation-in-part of application No. PCT/US2018/049373, filed on Sep. 4, 2018.
(60) Provisional application No. 62/553,532, filed on Sep. 1, 2017.
(60) Provisional application No. 62/615,330, filed on Jan. 9, 2018.
(60) Provisional application No. 62/615,433, filed on Jan. 9, 2018.
(60) Provisional application No. 62/664,722, filed on Apr. 30, 2018.

(51) Int. Cl. A61M 27/00; A61F 2/07; A61F 2/958; A61F 2/06

(52) U.S. Cl. CPC A61M 27/002; A61F 2/07; A61F 2/958; A61F 2/064; A61F 2250/0098; A61M 2210/127

(57) ABSTRACT

The disclosure provides various embodiments of prostheses and delivery systems to permit an interventional cardiologist to create shunts between various blood vessels. Moreover, the disclosed shunts can be used to shunt between various hollow organs, as set forth in the present disclosure.

### CROSS-REFERENCE TO RELATED APPLICATIONS

The present patent application is a continuation-in-part of and claims the benefit of priority to U.S. Pat. Appl. No. 16/264,402, filed Jan. 31, 2019, which in turn is a continuation of and claims the benefit of priority to International Application No. PCT/US2018/49373, filed Sep. 4, 2018, which in turn claims the benefit of priority to U.S. Provisional Patent Application Ser. No. 62/553,532, filed Sep. 1, 2017, U.S. Provisional Patent Application Ser. No. 62/615,330, filed Jan. 9, 2018, U.S. Provisional Patent Application Ser. No. 62/615,433, filed Jan. 9, 2018, and U.S. Provisional Patent Application Ser. No. 62/664,722, filed Apr. 30, 2018. The present patent application is also related to U.S. Patent Application Ser. No. 15/267,075, filed Sep. 15, 2016. Each of the foregoing patent applications is incorporated by reference herein for any purpose whatsoever.

### FIELD OF THE DISCLOSURE

The present disclosure relates to devices and methods for transcatheter (i.e., performed through the lumen of a catheter) Potts and related shunt systems for nonsurgical, percutaneous extra-anatomic bypass between two adjacent vessels and/or other anatomical structures.

### BACKGROUND

Various shunting procedures have been performed in open surgical procedures. The present disclosure is directed to devices and systems for performing shunting procedures by way of percutaneous approaches.

### SUMMARY OF THE DISCLOSURE

The purpose and advantages of the present disclosure will be set forth in and become apparent from the description that follows. Additional advantages of the disclosed embodiments will be realized and attained by the methods and systems particularly pointed out in the written description hereof, as well as from the appended drawings.

To achieve these and other advantages and in accordance with the purpose of the disclosure, as embodied herein, in one aspect, the disclosure includes embodiments of a percutaneously deliverable tubular prosthesis to permit an interventional cardiologist to create a shunt by way of a side to side anastomosis between the left pulmonary artery to the descending aorta, resulting in a right-to-left shunt. This is done to help decompress the right ventricle.

In some implementations, a tubular prosthesis is provided that includes an elongate compliant tubular body having a proximal end and a distal end, a distal sealing flange coupled to the distal end of the elongate compliant tubular body, the distal sealing flange being configured and arranged to facilitate seating the tubular prosthesis against a first concave vessel wall of a first vessel, wherein the tubular prosthesis is configured to extend outwardly through a first ostium formed in the first concave vessel wall when deployed. The distal sealing flange remains inside the first ostium after deployment. The tubular prosthesis can further include at least one laterally extending projection that is structurally distinct from the distal sealing flange. The at least one laterally extending projection is located proximate the distal sealing flange, and extends laterally beyond the distal sealing flange. The at least one laterally extending projection is configured and arranged to resist being pulled through the first ostium.

The aforementioned tubular prosthesis can further be provided with a proximal sealing flange coupled to the proximal end of the elongate compliant tubular body, the proximal sealing flange being configured and arranged to facilitate seating the tubular prosthesis against a second concave vessel wall of a second vessel, wherein the tubular prosthesis is configured to extend radially outwardly through a second ostium formed in the second concave vessel wall when deployed. The proximal sealing flange remains inside the second ostium after deployment. The tubular prosthesis can further include at least one laterally extending projection that is structurally distinct from the proximal sealing flange. The at least one laterally extending projection can be located proximate the proximal sealing flange, and extends laterally beyond the proximal sealing flange. The at least one laterally extending projection is configured and arranged to resist being pulled through the second ostium.

The at least one laterally extending projection on either end of the prosthesis can include two laterally extending projections that are oriented about 180 degrees with respect to each other about a longitudinal axis of the tubular prosthesis. The two laterally extending projections are preferably configured and arranged to rest near a bottom of the first concave vessel wall next to the ostium. Both laterally extending projections are configured and arranged to prevent the distal end of prosthesis from being pulled proximally through either ostium. The two laterally extending projections can be connected to a framework of the tubular prosthesis disposed proximally with respect to the distal sealing flange. For example, the two laterally extending projections can be integrated into a circumferential ring structure that forms a distal end portion of the prosthesis. The circumferential ring structure typically includes an undulating wire that circumferentially traverses a circumference of the tubular prosthesis. The undulating can be defined by a serpentine pattern along at least a part of its length that can have various shapes, such as a sinusoidal shape, a sawtooth shape, a curved wave shape, and the like. One or both of the laterally extending projections can be formed from the same undulating wire that forms the circumferential ring structure.

In some implementations, the circumferential ring structure is formed from an undulating wire that transitions from a serpentine pattern along a first circumferential face of the tubular prosthesis into a first of the two laterally extending projections, transitions from the first of the two laterally extending projections back into the serpentine pattern along a second circumferential face of the tubular prosthesis opposite to the first lateral side of the tubular prosthesis, transitions from the serpentine pattern into the second of the two laterally extending projections along the second circumferential face of the tubular prosthesis, and transitions from the second of the two laterally extending projections back to the serpentine pattern along the first circumferential face of the tubular prosthesis.

In some implementations of the tubular prosthesis, the membrane can be configured to covers the inside and/or outside of the elongate compliant tubular body and the distal flange. For example, the membrane can include a woven or non-woven fabric. If desired, the membrane can include an expanded polytetrafluoroethylene (“ePTFE”) material, and/or biological tissue material. If desired, the laterally extending projection(s) may, or may not be covered by the membrane. In some embodiments, the laterally extending projection(s) includes at least one radiopaque marker formed thereon. For example, each of the two diametrically opposed laterally extending projections can include at least one radiopaque marker formed thereon at a location that resides at either ostium during implantation near the base of each of the laterally extending projections. If desired, one or both of the two laterally extending projections further includes at least one radiopaque marker formed near an outward lateral tip of each of the two laterally extending projections, respectively. In some embodiments, the laterally extending projection(s) extend from a location proximal to the distal sealing flange to a location that is distal with respect to the distal sealing flange.

In some implementations of the tubular prosthesis, the distal and/or proximal sealing flange can be formed at least in part from an undulating, star-shaped circumferential wire frame that is structurally distinct from and located further within the ostium with respect to the circumferential ring structure. The undulating, star-shaped circumferential wire frame can be coupled to the circumferential ring structure. The undulating, star-shaped circumferential wire frame can be coupled to the circumferential ring structure by a plurality of fabric filaments, wherein the star-shaped circumferential wire frame of the flange is able to move with respect to the circumferential ring structure. If desired, the undulating, star-shaped circumferential wire frame of the flange can be coupled to the membrane (such as by stitching and/or adhesive or weaving), and further wherein the circumferential ring structure can be coupled to the membrane. The star-shaped circumferential wire frame of the flange can be configured to move or flex with respect to the circumferential ring structure.

In some embodiments, the elongate compliant tubular body can be formed from a plurality of longitudinally spaced undulating circumferential wire frames that are attached to a tubular membrane material. If desired, successive undulating circumferential wire frames (or strut rings) are circumferentially aligned so that they can nest along an axial direction to facilitate bending and shortening (axial collapse) of the prosthesis.

In some embodiments, the prosthesis can include a membrane that in turn includes an inner layer and an outer layer that cover the inner and outer surfaces of a framework of the prosthesis. In some implementations, the prosthesis can further include at least one elastic body that causes the tubular prosthesis to shorten in length when unconstrained. The at least one elastic body can include at least one tension coil spring that defines a lumen along its length. A central longitudinal axis of the at least one tension coil spring is preferably co-incident (or at least concentric) with a longitudinal axis of the prosthesis. Thus, the tubular prosthesis can be of adjustable telescoping length. Preferably, the inside diameter of the prosthesis remains substantially unchanged when the prosthesis is adjusted in length. The at least one tension coil spring can actually include a plurality of tension coil springs that may be adjacent to or concentrically located with respect to one another.

The disclosure further provides a delivery system including a prosthesis as described elsewhere herein mounted thereon, wherein the prosthesis is mounted on a longitudinal inner member and inside of a retractable sheath. The delivery system can further include at least one removable tether having a first end and a second end. The first and second ends of the tether can be routed through a portion of the prosthesis and extend proximally through and out of a proximal region of the delivery system. The delivery system can further include a first set of radiopaque markers near the distal end of the delivery system, and a second set of markers that are visible outside the patient during a procedure that indicates the relative position of the delivery system and prosthesis. The first and second set of markers can be configured to be maintained in registration with each other during the procedure. For example, the first set of markers can be located on a distal atraumatic tip of the delivery system made of iron oxide to facilitate navigation under MRI or other imaging modality to position the delivery system accurately, and wherein the second set of markers can indicate the relative longitudinal position of the portions of the delivery system. If desired, the markers can be configured to indicate when the distal sealing flange of the prosthesis is suitably configured to pull against an inner face of the wall of a lumen.

The disclosure further provides a delivery system that includes an elongate inner core member having a proximal end and a distal end, the distal end having a compliant atraumatic tip mounted thereon, an inflatable member mounted on the elongate inner core member, a prosthesis as described elsewhere herein mounted around the elongate inner core member, and a retractable sheath having a proximal end and a distal end. The retractable sheath is slidably disposed with respect to, and depending on its position along the elongate core member, selectively covers, the prosthesis and at least a part of the inflatable member. The delivery system can further include a first actuator configured and arranged to advance the sheath proximally with respect to the elongate inner core, inflatable member, and prosthesis, and, a second actuator coupled to a reservoir of fluid. The reservoir is fluidly coupled to the inflatable member, and actuating the second actuator causes the fluid to flow out of the reservoir into the inflatable member to cause the inflatable member to expand radially outwardly.

In some embodiments, the prosthesis is mounted at least partially over and surrounding the inflatable member. For example, a distal portion of the prosthesis can be mounted over the inflatable member, a proximal portion of the prosthesis can be mounted over the inflatable member, or a central portion of the prosthesis can be mounted over the inflatable member. If desired, the prosthesis can be mounted on the elongate inner core member proximally, or distally, with respect to the inflatable member.

In some embodiments, the compliant atraumatic tip can include a gradually tapering distal section that transitions from a larger proximal diameter to a smaller distal diameter. The compliant atraumatic tip can further include a gradually tapering proximal section that transitions from a smaller proximal diameter to a larger distal diameter. A distal end of the proximal section of the compliant atraumatic tip can abut a proximal end of the distal section of the compliant atraumatic tip.

The disclosure further provides methods of delivering and implanting a tubular prosthesis. The method includes providing a delivery system as described herein, delivering a distal end of the delivery system to a target location through the ostium of the first concave vessel wall, withdrawing the sheath proximally to expose the prosthesis, positioning the distal end of the prosthesis in the ostium so that the sealing flange and the at least one laterally extending projection are inside the first concave vessel wall and the elongate compliant tubular body extends through the ostium outside of the first vessel, actuating the second actuator to cause the inflatable member to expand, and expanding the distal end of the tubular prosthesis using the balloon to fit it into the ostium and to shape the sealing flange to fit against the first concave vessel wall.

If desired the inflatable member can be positioned distally with respect to the prosthesis, and the inflatable member can be inflated to outwardly flare the distal end of the prosthesis, as desired. The method can further include adjusting the length of the prosthesis to a desired length. The method can further include disposing a proximal end of the prosthesis inside of a second vessel. If desired, the proximal end of the prosthesis can be mounted transversely through a second ostium formed in a wall of the second vessel to shunt the first vessel to the second vessel.

It is to be understood that both the foregoing general description and the following detailed description are exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIGS. 1A-1B are views of a further embodiment of a structural frame portion of an embodiment of a prosthesis in accordance with the present disclosure.

FIGS. 1C-1D are views of still a further embodiment of a structural frame portion of an embodiment of a prosthesis in accordance with the present disclosure.

FIGS. 1E-1F are views of the embodiment of a structural frame portion of FIGS. 1C to 1D in situ across a piece of simulated tissue.

FIGS. 1G and 1H illustrate aspects of still a further embodiment of a prosthesis in accordance with the present disclosure.

FIG. 1I illustrates the structural frame portion of FIGS. 1G-1H stretched over a cylindrical mandrel.

FIG. 1J illustrates the structural frame portion of FIGS. 1G-1H stretched over a cylindrical mandrel and covered with a suitable membrane material.

FIGS. 1K-1M illustrates views of a distal end portion of a further delivery system for delivering a prosthesis in accordance with the present disclosure.

### DETAILED DESCRIPTION

Reference will now be made in detail to the present preferred embodiments of the disclosure, examples of which are illustrated in the accompanying drawings. The methods and corresponding steps of the disclosed embodiments will be described in conjunction with the detailed description of the systems. The exemplary embodiments illustrated herein can be used to perform Glenn, Fontan, and Pott shunting procedures as well as other types of shunting procedures, but in a percutaneous manner. It will be appreciated, however, that the disclosed embodiments, or variations thereof, can be used for a multitude of procedures involving the connection of blood vessels or other biological lumens to native or artificial structures. Such endograft devices represent a potential breakthrough for physicians and young patients who require a safe, less-burdensome, and effective alternative to open heart surgery: a percutaneous approach to heal congenital heart failure.

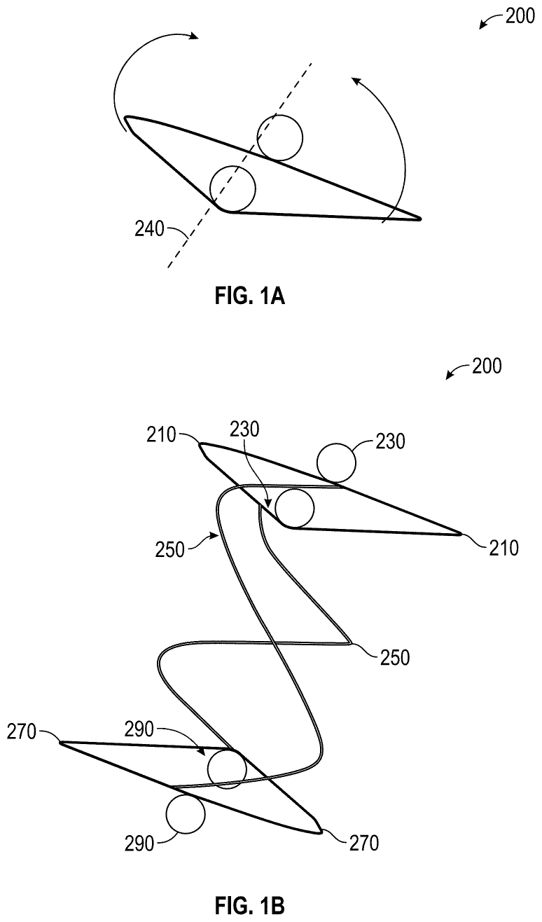

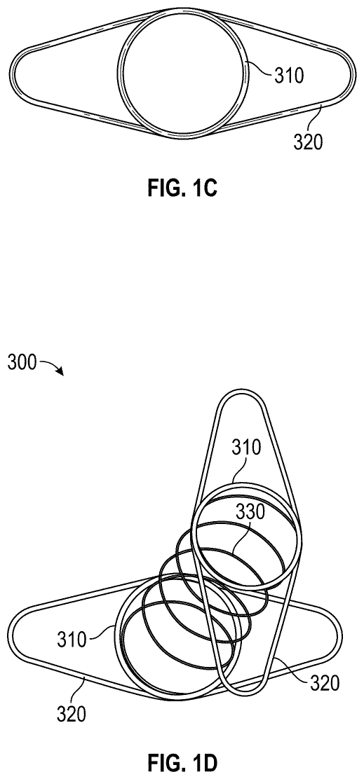

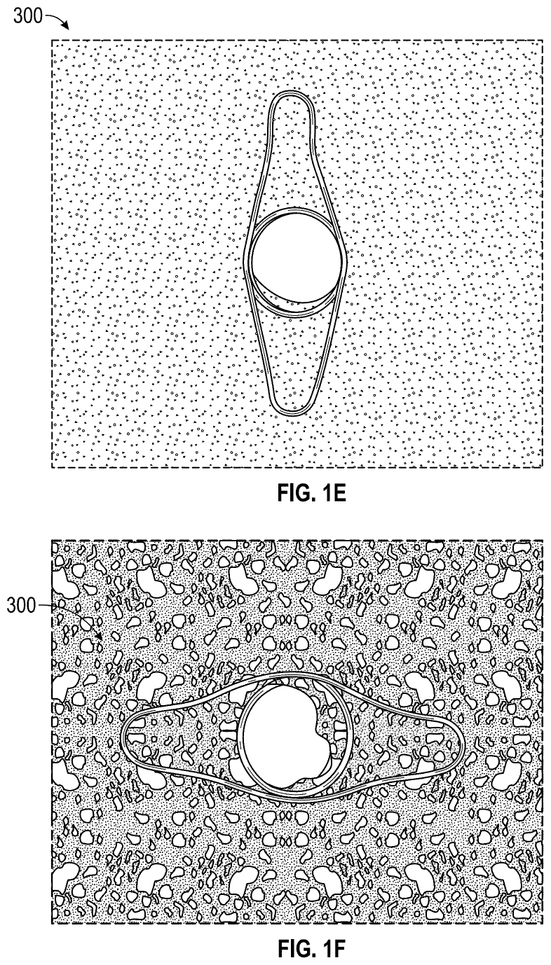

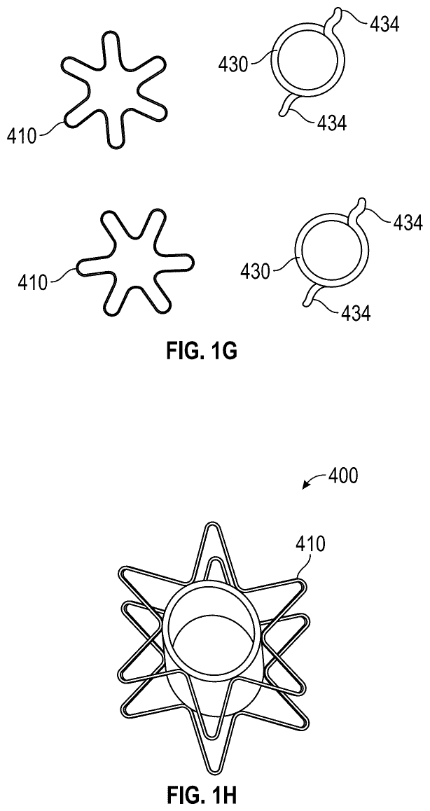

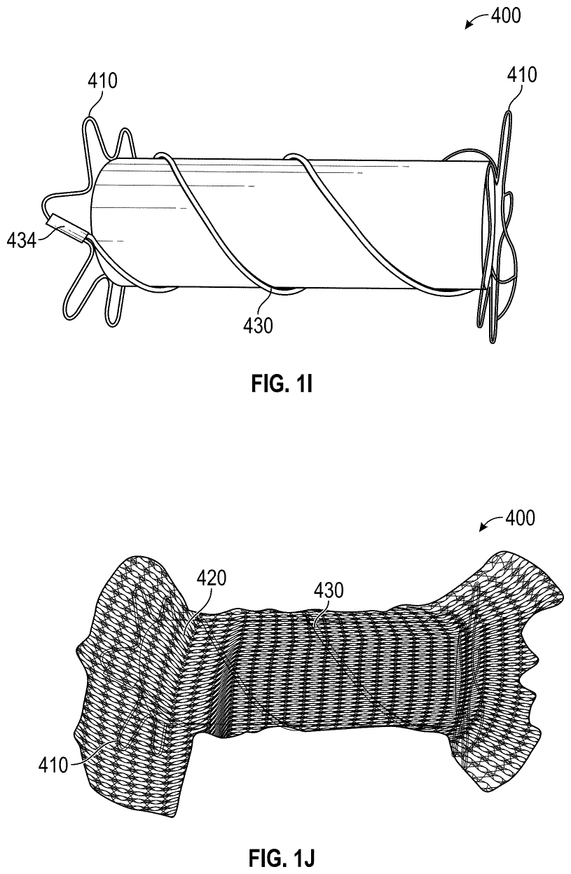

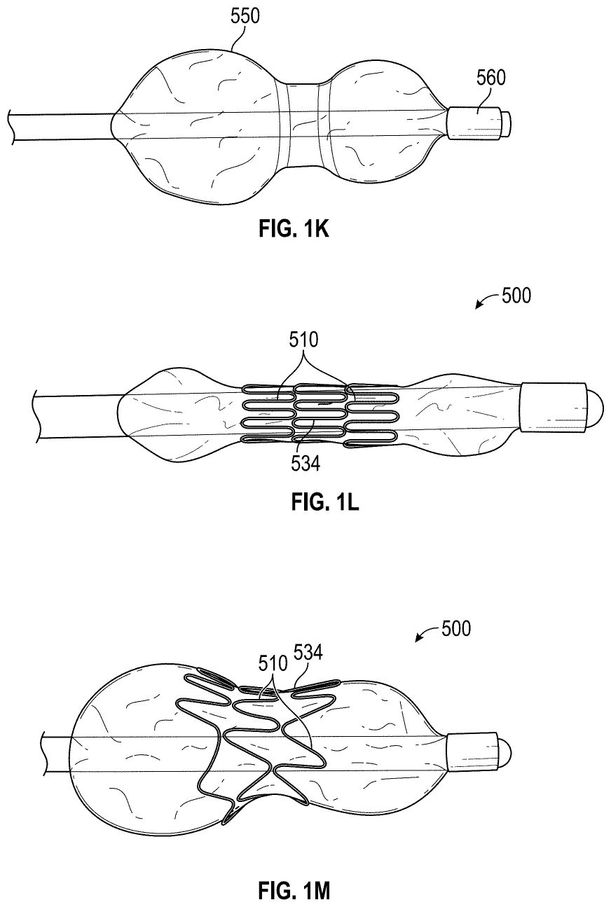

Embodiments of an axially collapsible prosthesis 200, 300, 400 are illustrated in FIGS. 1A-1H. These prostheses are generally similar in that they include an axially collapsible body that is typically defined by a helical spring, such as a tension spring, or similar member.

For purposes of illustration, FIGS. 1A-1B are views of a further embodiment of a structural frame portion of an embodiment of a prosthesis in accordance with the present disclosure. The example in FIG. 1A includes a collapsible prosthesis including folding lateral wings and a collapsible coil extending along the length of the prosthesis. As illustrated, each end of the prosthesis 200 includes folding lateral wings, 210 and 270. Folding lateral wings 210 are disposed on a first end of the prosthesis 200, and folding lateral wings 270 are disposed on a second end of the prosthesis opposite the first end, though folding lateral wings 210, 107-1, and 270 are structurally the same, but physically inverted with respect to each other, and if desired, rotationally aligned with each other about a longitudinal axis of the prosthesis 200. The folding lateral wings 210, 270 are configured to articulate orthogonally about an axis 240 via coils 230, 290. Coils 230 and wings 210/270, as illustrated are wound from the same strand of wire, such as NiTi alloy wire. Wings 210 fold inward towards one another by virtue of tension being wound into coils 230. This distributes the bending stress for the wings over a longer length of material, which can be advantageous as Ni Ti alloys tend to be brittle if bend over too short of a distance. In such a manner, folding lateral wings 210, in the folded state, may be compressed radially inwardly toward a central axis of the prosthesis 200 to facilitate reducing the profile of the prosthesis 200 to permit it to be collapsed and drawn into a delivery sheath of a delivery catheter. Folding lateral wings 270 are similarly configured to fold towards one another via folding, or “winding” coils 290 with tension.

As alluded to above, the folding lateral wings 210, 270, as well as the folding coils 230, can be comprised of a uniform heat formed wire, such as heat set nitinol, among other examples. For example, folding lateral wings 270, as well as folding coils 290 can be comprised of a uniform piece of wire heat shaped to extend laterally from the prosthesis in the uncompressed form, as illustrated in FIGS. 1A-1B. Each of the folding lateral wings 270, 290 may apply a force against a side wall of a vessel within which the prosthesis 200 is deployed, thereby preventing the prosthesis from being removed from an ostium formed through the vessel in a manner similar to wings/protrusions 132a discussed above. The end sections formed by wings 210/270 are also illustrated as being coupled to one or more (e.g, two or three) longitudinal coils 250. As illustrated, the collapsible coils 250 extend along a longitudinal length of the prosthesis, and couple the end sections to each other. As illustrated, each of the two coils 250 are out of phase with each other by about 180 degrees about a longitudinal axis of the prosthesis 200. In this manner, the coils 250 can structurally support inner and/or outer membrane layers to define a lumen through the prosthesis. Preferably, the coils 250 are evenly spaced from each other in this manner, such that two coils, as illustrated are spaced from each other about the axis, or out of phase, so to speak by 180 degrees, three coils are spaced from each other by 120 degrees, and four coils are spaced from each other by 90 degrees, and so on.

FIGS. 1C and 1D illustrate a further embodiment of a framework for a prosthesis 300. FIG. 1C illustrates structural supports of the end portions of the prosthesis 300. As illustrated, each end portion of prosthesis 300 includes an inner frame 310 coupled to an outer frame 320. Inner frame 310 and outer frame 320 can be made from the same piece of material wound about a mandrel (e.g., NiTi alloy wire) or different pieces of material that are attached to each other, for example, by soldering or welding. While inner frame 310 is circular, it will be appreciated that it may be other shapes, such as oval or polygonal. Outer frame 320, as presented, includes a widened central portion that aligns with the curvature of the inner frame 310 that tapers down on both sides to a projection, or wing, that is similar in function to wings 132b, 210, 270 described above, in that they are configured to prevent prosthesis 300 from being pulled through an ostium formed in a vessel wall. If desired, sealing flanges similar to those of FIG. 1A can be attached, for example to inner frame 310, extending toward the other end of the prosthesis, to provide a tapered sealing surface to fit into the ostium formed in the wall of a vessel or hollow organ.

As illustrated in FIG. 1D, the end frame portions, or flanges, of prosthesis 300 can be connected to each other by one or more coil springs in the same manner as prosthesis 200. While only one spring 330 is shown, it will be appreciated that multiple coil springs that can be used that are of different overall diameters such that they can nest inside one another. If desired, the springs 330 can additionally or alternatively be rotationally spaced from each other evenly or unevenly about a central longitudinal axis of the prosthesis 300. If desired, the end flanges of prosthesis 300 can additionally or alternatively be connected by strut rings and membrane material in a manner similar to the embodiment of FIGS. 1A-1D. As illustrated, the end flanges (310, 320) of prosthesis are rotated 90 degrees with respect to one another about a longitudinal axis of the prosthesis 300. As will further be appreciated, regardless as to the structural framework of prosthesis 300, prosthesis 300 preferably includes inner and/or outer membrane, or fabric, layers. FIGS. 1E and 1F illustrate the framework of prosthesis 300 deployed in a thick piece of material intended to simulate tissue, such as two nearby blood vessels to be shunted to each other.

FIG. 1G illustrates components an embodiment of a prosthesis 400 in accordance with the present disclosure illustrated in various stages of assembly in FIGS. 1H, 1I and 1J. Prosthesis includes a structural frame portion including proximal and distal flanges 410 connected to each other by one or more (e.g., two) tension coil springs 430. Similar to the embodiment of FIG. 1A, prosthesis 400 includes proximal and distal sealing flanges that are preferably at least partially covered in fabric or other membrane 420 (FIG. 1J). The coil springs 430 each include two terminal projections 434 that are attached to radially oriented portions of flanges 410, for example, by way of soldering or welding, to provide a strong joint. Multiple coils that are evenly or unevenly spaced that can nest within each other can be provided as described with respect to prosthesis 300 illustrated hereinabove. FIG. 1H illustrates an end view of prosthesis 400 clearly showing flange 410. If desired, one or more marker bands can be provided on flange 410. Alternatively, flange 410 and/or coil spring(s) 430 can be made from radiopaque material. Membrane material 420 can be provided inside, outside, and/or in between coil springs 430 for prosthesis 400. Also, if desired, strut rings can be substituted for coil springs 430 in prosthesis 400 as with the embodiment of FIG. 1A.

FIGS. 1K-1M illustrate additional embodiments of a collapsible prosthesis 500, in accordance with the present disclosure. FIGS. 1A-1D illustrate a collapsible prosthesis 500 including proximal and distal flanges 510 attached to each other by an undulating strut ring 534, similar to those described with respect to FIG. 1A. Prosthesis can be crimped onto a distally formed balloon 550 that is in turn mounted to an elongate inner member 560 of a delivery system. Prosthesis 500 can be collapsed radially inwardly (e.g., by crimping) onto balloon 550. As illustrated in FIGS. 1K-1M, a dual-lobed balloon including a proximal bulb and a distal bulb connected by a neck portion may be used to expand and outwardly flare the flanges 510 of prosthesis 500, for example, to form a shunt between two nearby vessels. The dual-lobed balloon can be formed from separate inflatable balloons, or a singular inflatable enclosure with a narrowed neck as illustrated. Prosthesis 500 is preferably provided with an inner and/or outer membrane covering (not shown). FIG. 1K illustrates the balloon in an inflated condition, FIG. 1L illustrates the prosthesis (illustrating the frame only) 500 crimped on the balloon prior to delivery and FIG. 1M shows the prosthesis 500 in a partially deployed condition by virtue of inflating the balloon. Such a balloon with multiple lobes, or proximal and distal neck regions and a larger central lobe can be used to selectively flare ends of prostheses as described below.

In general, it will be appreciated that any of the prostheses disclosed herein can further include at least one elastic body (e.g., tension coil spring) that causes the tubular prosthesis to shorten in length when unconstrained. The at least one elastic body can include at least one tension coil spring that defines a lumen along its length. A central longitudinal axis of the at least one tension coil spring is preferably co-incident (or at least concentric) with a longitudinal axis of the prosthesis. Thus, the tubular prosthesis can be of adjustable telescoping length. Preferably, the inside diameter of the prosthesis remains substantially unchanged when the prosthesis is adjusted in length. The at least one tension coil spring can actually include a plurality of tension coil springs that may be adjacent to or concentrically located with respect to one another.

The disclosure further provides a delivery system including a prosthesis as described elsewhere herein mounted thereon, such as illustrated in part in FIGS. 1K-1M. As shown in part, the prosthesis can be mounted on a longitudinal inner member of a delivery system. The delivery system can thus include an elongate inner core member having a proximal end and a distal end. The distal end can have a compliant atraumatic tip mounted thereon that may have a gradual distal taper, and may also include a proximal taper, if desired, similar to the delivery system illustrated in FIGS. 4A-4H of U.S. Pat. Appl. No. 16/264,402 to ease removal of the distal end of the delivery system from a blood vessel back into a shunt that has been mounted as the delivery system is being withdrawn.

If desired, the delivery system can include an inflatable member mounted on the elongate inner core member, and the prosthesis can be mounted around the elongate inner core member. A retractable sheath can also be provided having a proximal end and a distal end. The retractable sheath can be slidably disposed with respect to, and depending on its position along the elongate core member, can selectively cover the prosthesis and at least a part of the inflatable member. The delivery system can further include a first actuator (not shown) configured and arranged to advance the sheath proximally with respect to the elongate inner core, inflatable member, and prosthesis. A second actuator can be coupled to a reservoir of fluid. The reservoir can be fluidly coupled to the inflatable member, and actuating the second actuator can cause the fluid to flow out of the reservoir into the inflatable member to cause the inflatable member to expand radially outwardly. Specifically, for purposes of illustration, FIG. 1M shows a distal portion of the delivery system showing a partially deployed prosthesis located over and surrounding a balloon used for inflation, whereas FIG. 1L shows the uninflated, elongate balloon with the prosthesis being present.

As mentioned above, in some embodiments, the prosthesis can be mounted at least partially over and surrounding the inflatable member. For example, a distal portion of the prosthesis can be mounted over the inflatable member, a proximal portion of the prosthesis can be mounted over the inflatable member, or a central portion of the prosthesis can be mounted over the inflatable member. If desired, the prosthesis can be mounted on the elongate inner core member proximally, or distally, with respect to the inflatable member.

An exemplary method in accordance with the disclosure includes providing a delivery system as described herein, delivering a distal end of the delivery system to a target location through the ostium of the first concave vessel wall, withdrawing the sheath proximally to expose the prosthesis, positioning the distal end of the prosthesis in the ostium so that the sealing flange and the at least one laterally extending projection are inside the first concave vessel wall and the elongate compliant tubular body extends through the ostium outside of the first vessel, actuating the second actuator to cause the inflatable member to expand, and expanding the distal end of the tubular prosthesis using the balloon to fit it into the ostium and to shape the sealing flange to fit against the first concave vessel wall.

If desired the inflatable member can be positioned distally with respect to the prosthesis, and the inflatable member can be inflated to outwardly flare the distal end of the prosthesis, as desired. The method can further include adjusting the length of the prosthesis to a desired length. The method can further include disposing a proximal end of the prosthesis inside of a second vessel. For example, the proximal end of the prosthesis can be mounted transversely through a second ostium formed in a wall of the second vessel to shunt the first vessel to the second vessel.

Pulmonary hypertension of diverse etiologies causes severe symptoms and high mortality rate. Symptoms include inability to exercise, shortness of breath, right-sided congestive heart failure, and sudden death. New pharmacologic options have significantly prolonged survival in adults with severe pulmonary hypertension. These therapeutic options have led to nationwide centers of excellence for the care of pulmonary hypertension. Despite successful pharmacotherapy, the disease progresses in the majority causing progressive right ventricular failure and declining functional status. Heart-lung transplantation may not be an option.

Forming a “Potts” shunt (between the left pulmonary artery and the descending thoracic aorta) is a surgical procedure that can divert blood flow to relieve right heart failure in patients with end-stage pulmonary hypertension. It can be offered as a bridge to transplantation or as a destination therapy. Surgical Potts shunt is morbid and complex. In accordance with the present disclosure, a catheter-based Potts shunt (such as that illustrated in FIGS. 1A-1J can be delivered by way of a delivery system as set forth herein and used to shunt the left pulmonary artery to the descending thoracic aorta.

If desired, in some embodiments, the proximal end of the prosthesis (e.g., 200, 300, 400, 500) can receive a tether therethrough that is routed through the windings of the most proximal undulating strut ring through openings defined in membrane material. The tethers are withdrawn proximally through a tubular member (e.g., a sheath) that also passes a core member therethrough that forms the core, or push rod of the delivery system. The core is slidably disposable with respect to the sheath. By advancing the core member with the prosthesis mounted thereto distally outwardly of the sheath, the prosthesis can self-expand, or be expanded by a balloon. However, if the tether is tensioned, it can cause the proximal end of the prosthesis to collapse radially inwardly such that the prosthesis can be withdrawn into the sheath. While adjacent undulating rings of the prosthesis particularly near the distal end of the prosthesis can be connected to each other (e.g., by sewing), they can also be kept independent of one another, and be attached to an inner and/or outer tubular fabric layer. The rigidity of the prosthesis is selected and/or configured to provide a desired performance. Thus, the distal end can be relatively rigid to maintain an opening in the wall of a vessel or other organ in an open state that the prosthesis traverses through by resisting the force of the vessel wall to want to “close” the hole in itself. The proximal region is less rigid and can accommodate increasing vessel curvature of the vessel that it is mounted in.

The devices and methods disclosed herein can be used for other procedures in an as-is condition, or can be modified as needed to suit the particular procedure. In view of the many possible embodiments to which the principles of this disclosure may be applied, it should be recognized that the illustrated embodiments are only preferred examples of the disclosure and should not be taken as limiting the scope of the disclosure.

What is claimed is:

1. A method of delivering a tubular prosthesis in a Potts procedure, comprising:
providing a prosthesis on a percutaneous delivery catheter including:
an elongate compliant tubular body having a proximal end and a distal end;
a distal sealing flange coupled to the distal end of the elongate compliant tubular body, the distal sealing flange being configured and arranged to facilitate seating the tubular prosthesis against a first concave vessel wall of a first vessel, wherein the tubular prosthesis is configured to extend outwardly through a first ostium formed in the first concave vessel wall when deployed, wherein the distal sealing flange remains inside the ostium after deployment;
a proximal sealing flange coupled to the proximal end of the elongate compliant tubular body, the proximal sealing flange being configured and arranged to facilitate seating the tubular prosthesis against a second concave vessel wall of a second vessel, wherein the tubular prosthesis is configured to extend outwardly through a second ostium formed in the second concave vessel wall when deployed, wherein the distal sealing flange remains inside the second ostium after deployment;
introducing the prosthesis on the percutaneous delivery catheter into a patient's vasculature;
forming an ostium into a wall of the patient's left pulmonary artery and an ostium into a wall of the patient's descending aorta;
deploying a first of the proximal sealing flange and distal sealing flange into the ostium formed into the wall of the left pulmonary artery; and
deploying the other of the proximal sealing flange and distal sealing flange into the ostium formed into the wall of the patient's descending aorta.

2. The method of claim 1, wherein at least one of the proximal and distal ends of the prosthesis further includes at least one laterally extending projection structurally distinct from the distal and proximal sealing flanges, the at least one laterally extending projection being located proximate the distal or proximal sealing flange and extending laterally beyond the distal or proximal sealing flange, respectively, the at least one laterally extending projection being configured and arranged to resist being pulled through said wall of said aorta or left pulmonary artery.

3. The method of claim 2, wherein the at least one laterally extending projection includes at least two laterally extending projections oriented about 180 degrees with respect to each other about a longitudinal axis of the tubular prosthesis, and further wherein the at least two laterally extending projections are configured and arranged to rest near a bottom of a concavity of the left pulmonary artery or right descending aorta.

4. The method of claim 3, wherein the at least two laterally extending projections are connected to a framework of the tubular prosthesis, and extend radially outwardly with respect to the proximal sealing flange or the distal sealing flange and extend further into a respective blood vessel than the proximal sealing flange or distal sealing flange.

5. The method of claim 4, wherein the at least two laterally extending projections are integrated into a circumferential ring structure that forms a proximal or distal end portion of the prosthesis.

6. The method of claim 5, wherein the circumferential ring structure includes an undulating wire that circumferentially traverses a circumference of the tubular prosthesis, the undulating wire being defined by a serpentine pattern along at least a part of its length.

7. The method of claim 5, wherein at least one of the at least two laterally extending projections is formed from the same undulating wire that forms the circumferential ring structure.

8. The method of claim 6, wherein two laterally extending projections are formed from the same undulating wire that forms the circumferential ring structure.

9. The method of claim 6, wherein the circumferential ring structure is formed from an undulating wire that:
transitions from a serpentine pattern along a first circumferential face of the tubular prosthesis into a first of the two laterally extending projections;
transitions from the first of the two laterally extending projections back into the serpentine pattern along a second circumferential face of the tubular prosthesis opposite to the first lateral side of the tubular prosthesis;
transitions from the serpentine pattern into the second of the two laterally extending projections along the second circumferential face of the tubular prosthesis; and
transitions from the second of the two laterally extending projections back to the serpentine pattern along the first circumferential face of the tubular prosthesis.

10. The method of claim 1, wherein a membrane covers the elongate compliant tubular body and the distal flange and proximal flange.

11. The method of claim 10, wherein the membrane includes a woven or non-woven fabric.

12. The method of claim 10, wherein the membrane includes an expanded polytetrafluoroethylene (“ePTFE”) material.

13. The method of claim 10, wherein the membrane includes a biological tissue material.

14. The method of claim 10, wherein the at least two laterally extending projections are not covered by the membrane.

15. The method of claim 10, wherein each of the at least two laterally extending projections includes at least one radiopaque marker formed thereon.

16. The method of claim 15, wherein each of the at least two laterally extending projections includes at least one radiopaque marker formed thereon at a location that resides at a respective ostium after implantation.

17. The method of claim 15, wherein each of the at least two laterally extending projections further includes at least one radiopaque marker formed near an outward lateral tip of each of the two laterally extending projections, respectively.
